(19) United States
(12) Patent Application Publication
(10) Pub. No.: US 2021/0289724 A1
(43) Pub. Date: Sep. 23, 2021

(54) SMART GARDEN STATIONS

(71) Applicant: Sergio Prado Chavez, Ciudad de Mexico (MX)

(72) Inventor: Sergio Prado Chavez, Ciudad de Mexico (MX)

(21) Appl. No.: 16/821,327

(22) Filed: Mar. 17, 2020

(51) Int. Cl. A01G 27/00; A01G 25/16

(52) U.S. Cl. CPC A01G 27/005; A01G 27/008; G01N 33/246; A01G 27/003; A01G 25/167

(57) ABSTRACT

A smart garden station for remotely managing growth and health of indoor plants is disclosed. The smart garden station comprises of a first container mounted on a panel and a second container inserted into the first container onto which a growing medium is disposed for planting seeds/plants. A set of sensors constantly monitor and communicate environment related data around the garden station to a remote electronic device. The remote electronic device generates a set of instructions to provide ideal environmental conditions at the garden station based on the received data. A processing unit receives these instructions and manages environmental conditions around the garden station. The processing unit controls the environment by primarily controlling the watering unit, the lighting unit, and the ventilation unit. These components modulate water content in growing medium and the temperature/humidity around the garden station to provide an optimal environment for the growth and health of the plants.

### TECHNICAL FIELD

The present disclosure relates to techniques for providing portable smart gardens and more particularly to methods, techniques, and apparatuses for developing a portable self-sustaining garden station that can be monitored and controlled by electronic devices.

### BACKGROUND

Indoor planting and cultivation techniques have been practised by humans for centuries, such plants absorb Carbon dioxide and keep oxygen flowing. Further, they purify the air by removing toxins, help to deter illness, ease tension and lower stress, create a relaxed and happy ambience. The air purifying and filtering properties of indoor plants ultimately foster a healthier and happier working and living environment. Vertical growing on supporting structures, wall plants, potted plants are some of the popular examples of plants being grown indoors.

Indoor plants are generally referred to as houseplants. While they are generally grown for decorative purposes by many, studies have also shown them to have positive psychological effects and as well as help with indoor air purification, since some species, and the soil-dwelling microbes associated with them, reduce indoor air pollution by absorbing volatile organic compounds including benzene, formaldehyde, and trichloroethylene. While generally toxic to humans, such pollutants are absorbed by the plant and its soil-dwelling microbes without harm.

Houseplants need the correct moisture, light levels, soil mixture, temperature, and humidity for proper growth, such conditions generally keep the plant healthy. Major factors that should be considered when caring for houseplants are moisture, light, soil mixture, temperature, humidity, fertilizers, potting, and pest control.

Both under-watering and over-watering can be detrimental to a houseplant. Different species of houseplants require different soil moisture levels. Brown crispy tips on a plant's leaves are a sign that the plant is under-watered. Yellowing leaves can show that the plant is over watered. House plants are generally planted in pots that have drainage holes in the bottom of the pot to reduce the likelihood of over watering and standing water. Most plants cannot withstand their roots sitting in water and will often lead to root rot.

Different plants require different amounts of light, for different duration. It has been observed that the growth of some plants are influenced either by decreasing or increasing daylight hours. It is possible to supplement window light with artificial lighting of suitable wavelengths.

Soil is another important factor for growing healthy plants indoors. The moisture content, quality, nutrients, aeration, etc., need to be monitored and adjusted constantly to maintain the quality of soil. Similarly, temperature is another criterion for plants to grow and stay healthy. Most plants have a defined range of home temperature within which they thrive the best. Hence, ambient temperature depending on the plant species is a crucial factor.

Humidity is slightly more difficult to control than temperature. Homes often have around 20% to 60% relative humidity. While such range may work for some species, most species thrive near 80% relative humidity. Indoor plants also give off moisture into the air generally raising the relative humidity in the room.

The phenomenon of biophilia explains why houseplants have positive psychological effects on humans. Biophilia describes humans' subconscious need for a connection with nature. This is why humans are fascinated by natural spectacles such as waves or a fire. It also explains why gardening and spending time outdoors can have healing effects. Having plants in indoor living areas can have positive effects on physiological, psychological and cognitive health. There is an architectural design approach known as “complexity and order” that coincides with biophilic design. Complexity and order is defined as, “the presence of rich sensory information that is configured with a coherent spatial hierarchy, similar to the occurrence of design in nature.” Humans enjoy looking at things that are not boring but also not too complex. This design strategy is based on nature and human's response to designs in nature. The presence of plants and nature-inspired designs is restorative and not dull like the modern cookie cutter designs.

While comprehensive research show that growing plants indoors is advantageous to both physical and mental health of humans, it has become challenging to manage such plants due to constraints caused by modern lifestyle. Many a time, taking care of such indoor plants become cumbersome, this affects the growth and health of such plants. Hence, it may be advantageous to provide a modern garden station that may be automated using electronic and mechanical components.

### BRIEF DESCRIPTION OF THE DRAWINGS

Examples are described in the following detailed description and in reference to the drawings, in which:

FIG. 1A is an illustration of the first and second containers according to the embodiment of the present disclosure;

FIG. 1B is another depiction of the first and second containers of FIG. 1A according to the embodiments of the present disclosure;

FIG. 2A is a cross-sectional view of the first and second containers showing the watering base and the watering unit according to the embodiment of the present disclosure;

FIG. 2B is another depiction of the cross-sectional view of the first and second containers for FIG. 2A according to the embodiment of the present disclosure;

FIG. 3A is an illustration of the second container according to the embodiment of the present disclosure;

FIG. 3B is another depiction of the second container of FIG. 3A according to the embodiment of the present disclosure;

FIG. 4A is another illustration of the second container showing its top and bottom openings according the embodiments of the present disclosure;

FIG. 4B is another depiction of the second container of FIG. 4A according to the embodiment of the present disclosure;

FIG. 5A is an illustrative top view of the watering base according to the embodiment of the present disclosure;

FIG. 5B is another depiction of the watering base of FIG. 5A according to the embodiment of the present disclosure;

FIG. 6A is an illustration of the bottom view of the second container, watering base, and the watering unit according to the embodiment of the present disclosure:

FIG. 6B is another depiction of the FIG. 6A according to the embodiment of the present disclosure;

FIG. 7A is an exemplary view of the base structure of the panel of the smart garden station according to the embodiment of the present disclosure;

FIG. 7B is an exemplary view of the panel of the smart garden station according to the embodiment of the present disclosure;

FIG. 8A is an exemplary depiction of the panel of the smart garden station with power ports, ventilation unit, and lighting unit interfaced to it according to the embodiment of the present disclosure;

FIG. 8B is an exemplary rear view of the smart garden station of FIG. 8A according to the embodiment of the present disclosure;

FIG. 9 is an exemplary depiction of a smart garden station with two container pots according to the embodiment of the present disclosure;

FIG. 10 is an exemplary depiction of a smart garden station with a single container pot according to the embodiment of the present disclosure;

FIG. 11A is a side view of a power port used in the smart garden station according to the embodiment of the present disclosure;

FIG. 11B is another view of the power port of FIG. 11A according to the embodiment of the present disclosure;

FIG. 12 is a block diagram of the architecture of the smart garden station management unit according to the embodiment of the present disclosure.

The drawings described herein are for illustration purposes only and are not intended to limit the scope of the present subject matter in any way.

### DETAILED DESCRIPTION

Embodiments described herein discloses methods, techniques, and apparatuses for developing a portable garden station that can be monitored and controlled remotely by electronic devices. Embodiments described herein discloses an apparatus that may be used as a smart portable garden station for germinating and growing plants in a controlled environment. The apparatus comprises of a plurality of functional components that may be connected together to resemble a smart portable garden station. The functions of the apparatus may be monitored and managed by an electronic device that is communicatively connected to the apparatus.

The primary components of the apparatus include pot-shaped containers that may be used for germinating and growing a plant, a panel onto which the container pot is mounted upon and interfaced with the power ports, a lighting unit that may provide adequate light for the plants, a set of fans for providing ventilation to the plants, a motor for watering the plants, a plurality of sensors to continuously monitor parameters such as light, temperature, moisture, humidity, movement, and the like; a processing unit and an associated memory unit that manages the overall functioning of the apparatus, wireless communication modules for communicating with external devices, power connectors and allied ports, a set of power supply connectors, and an exterior supporting structure capable of being mounted onto standard television support frames.

The functioning of various components of the apparatus may be controlled and managed using a software application installed in an electronic device (such as a smartphone). The apparatus may be communicatively coupled to the electronic device via wireless communication means. The application installed in the electronic device may allow a user to control watering, lighting, humidity, and ventilation related parameters. Furthermore, the application may allow users to track the growth of the plant and the germination process. A user may manually control the application or may automate the application to run as per a fixed schedule or criterion. The apparatus may allow users to remotely manage the environment provided to the plants and track the growth of the plants via the electronic device.

In the following sections, some features are grouped together in a single embodiment for streamlining the disclosure. This method of disclosure is not to be interpreted as reflecting an intention that the disclosed embodiments of the present disclosure must use more features than are expressly recited in each claim. Rather, as the following claims reflect, inventive subject matter lies in less than all features of a single disclosed embodiment. Thus, the claims are hereby incorporated into the detailed description, with each claim standing on its own as a separate embodiment.

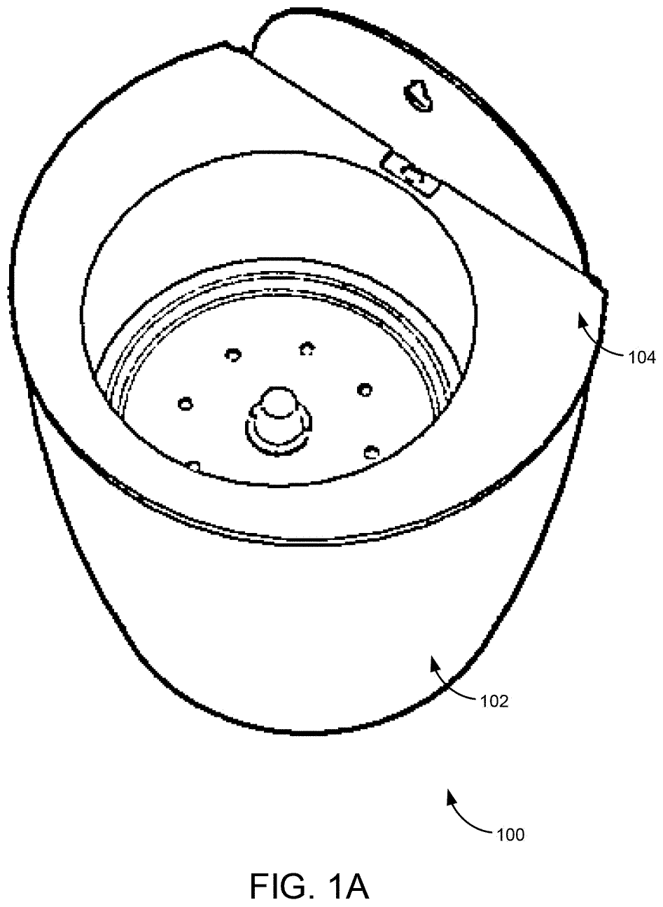

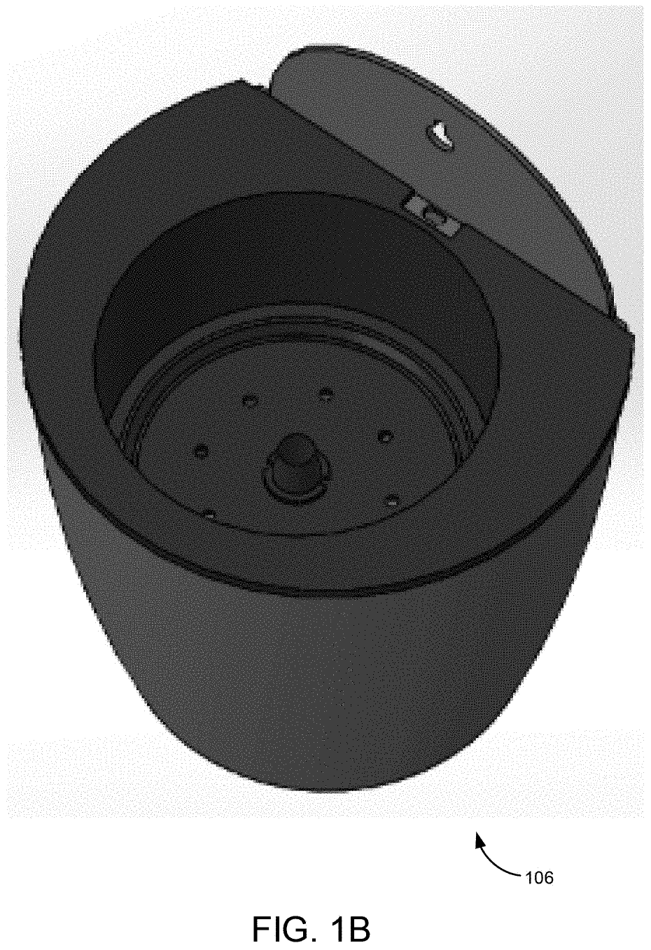

Referring to figures, FIG. 1A is an illustration 100 of the first and second containers according to the embodiment of the present disclosure. The first container is depicted by 102 and the second container is depicted by 104. The first container 102 may be closed on all sides except its top side and may be shaped like a pot. The second container 104 may be inserted into the first container and may fill the top portion of the first container 102. It may be noted that the depth of the second container 104 may be lesser than that of the first container 102, hence the bottom portion of the first container 102 may have an empty space when the second container 104 is inserted into the first container 102. Moreover, the top edge of the second container has a wider shape which acts as a stopper when the second container 104 is inserted into the first container 102. Furthermore, the second container 104 may be open on its top side and partially open on its bottom side. FIG. 1B is another depiction 106 of the first and second containers of FIG. 1A according to the embodiments of the present disclosure. The structure and design of the containers can be understood in more detail when referring to the following figures.

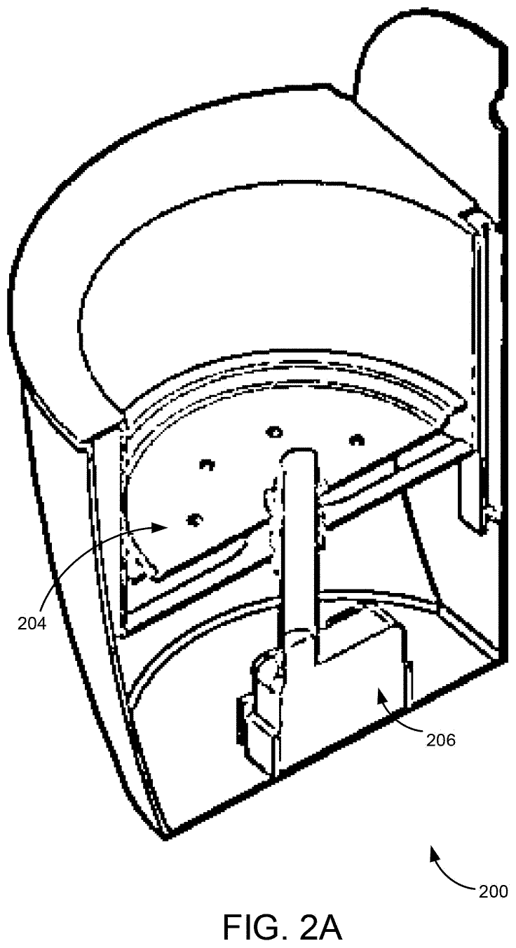

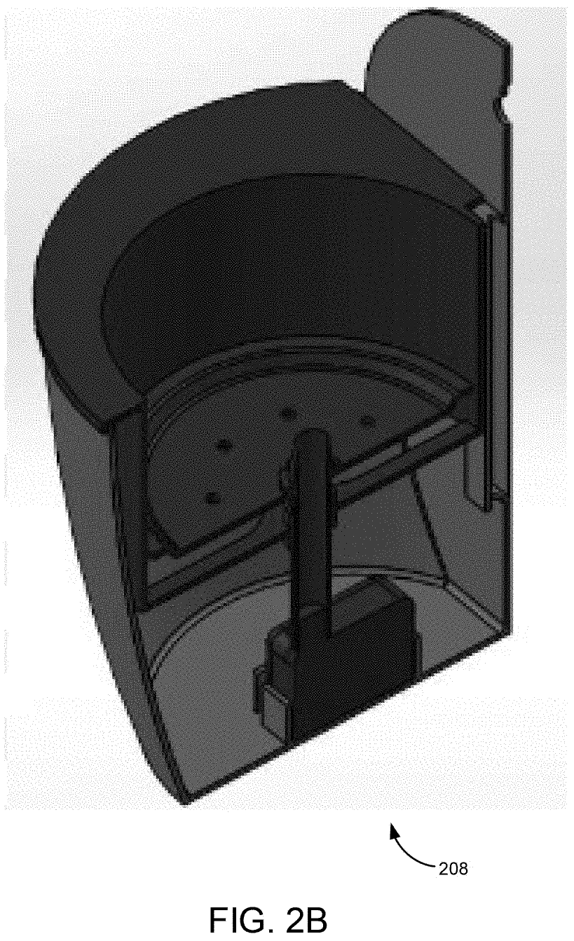

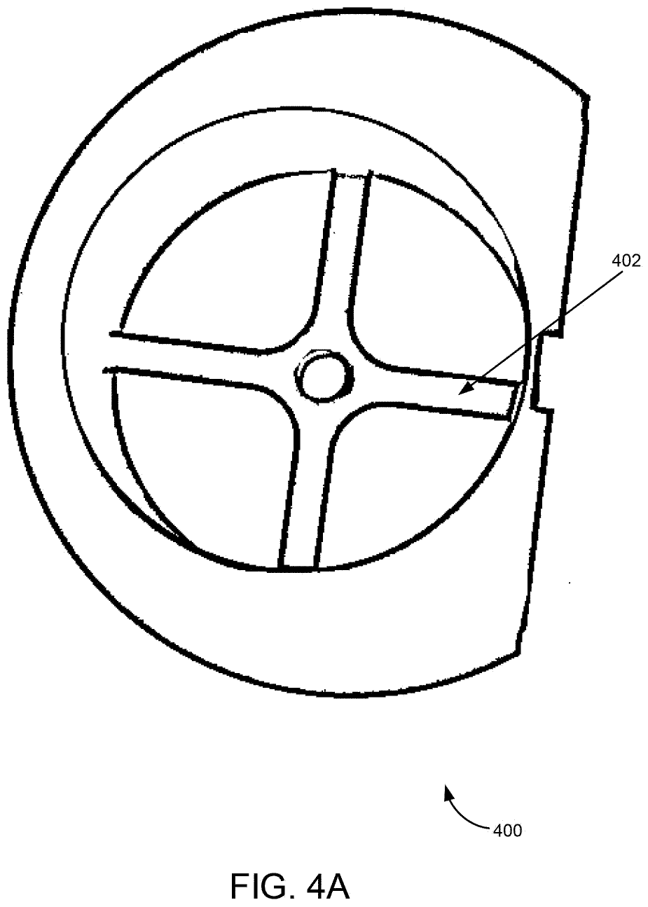

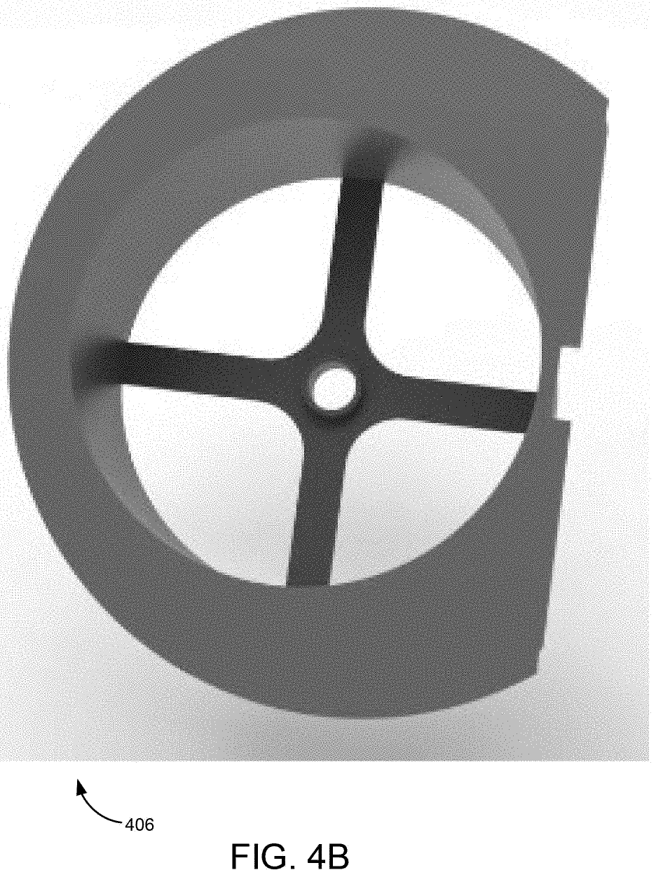

FIG. 2A is a cross sectional view 200 of the first and second containers showing the watering base and the watering unit according to the embodiment of the present disclosure. The bottom side of the second container 104 is partially open (shown in more detail in FIGS. 4A and 4B), the bottom side of the second container 104 may be connected with a watering base 204. The watering 204 base may have a plurality of openings and may be connected to a watering unit 206 via a tube. The watering unit 206 may be installed in the empty space at the bottom portion of the first container 102 as depicted in the figure. FIG. 2B is another depiction 208 of the cross-sectional view of the first and second containers for FIG. 2A according to the embodiment of the present disclosure.

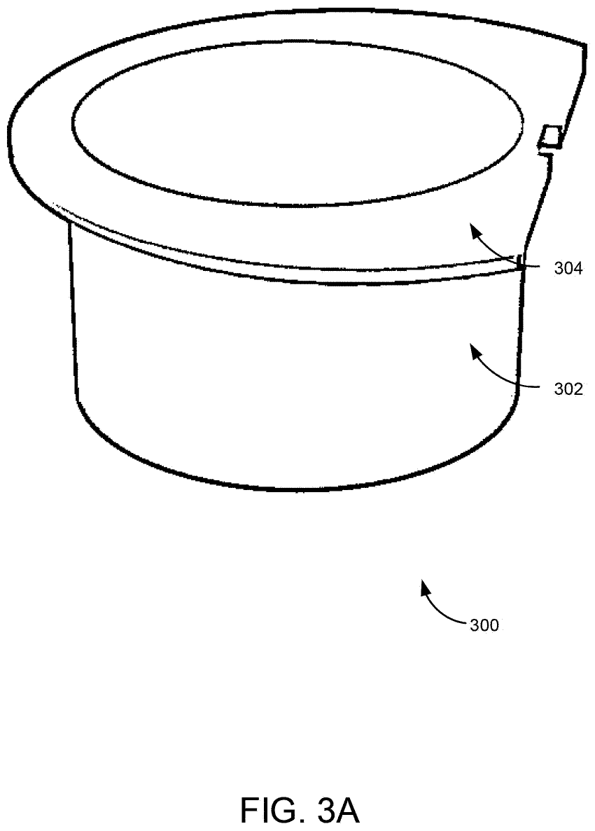

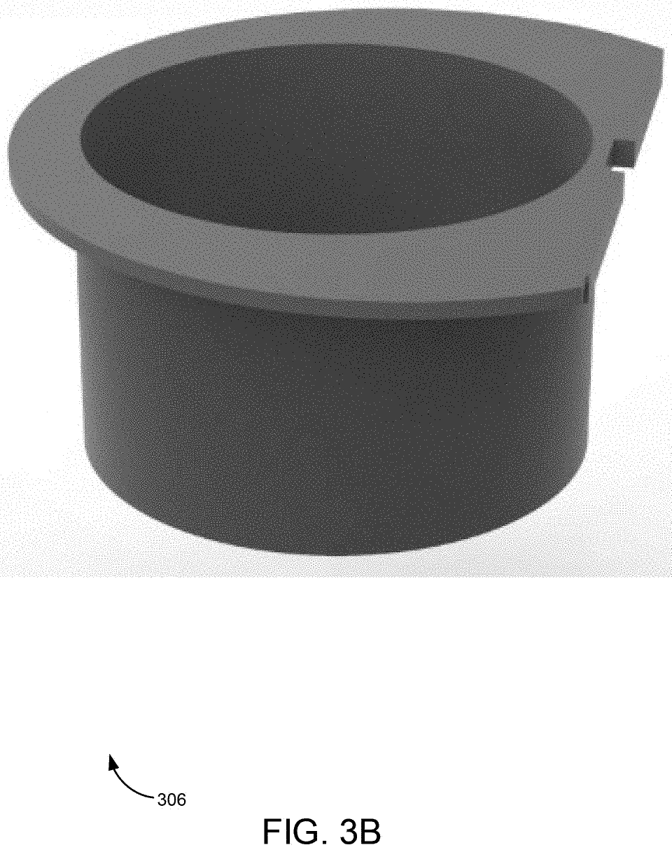

FIG. 3A is an illustration 300 of the second container according to the embodiment of the present disclosure. The body 302 of the second container 104 has a smaller circumference compared to the circumference of the first container 102 while the top side has a wider edge 304 that acts as a stopper when it is inserted into the first container 102. FIG. 3B is another depiction 306 of the second container of FIG. 3A according to the embodiment of the present disclosure. FIG. 4A is another illustration 400 of the second container showing its top and bottom openings according the embodiments of the present disclosure. While the top side is completely open, the bottom side is partially open. There is a stopper like mechanism 402 at the bottom side. FIG. 4B is another depiction 406 of the second container of FIG. 4A according to the embodiment of the present disclosure.

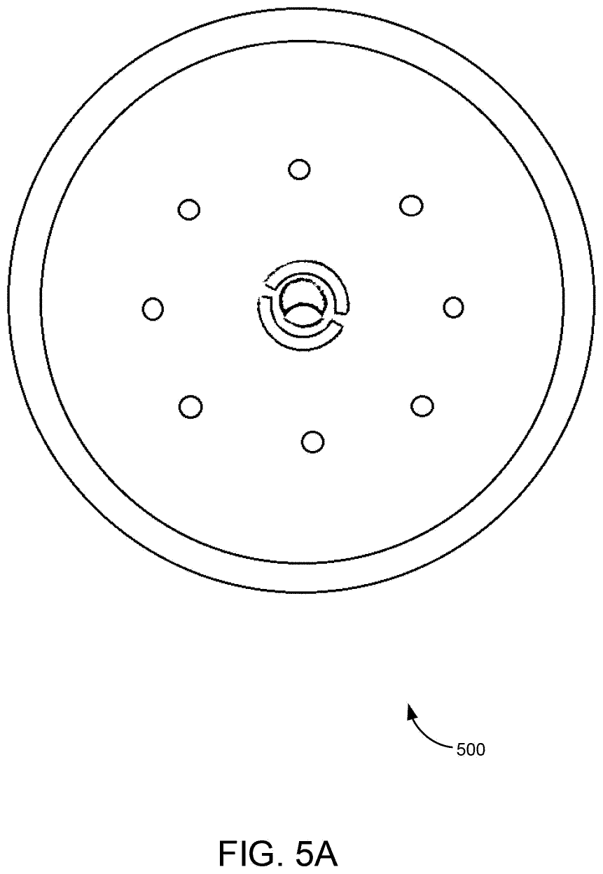

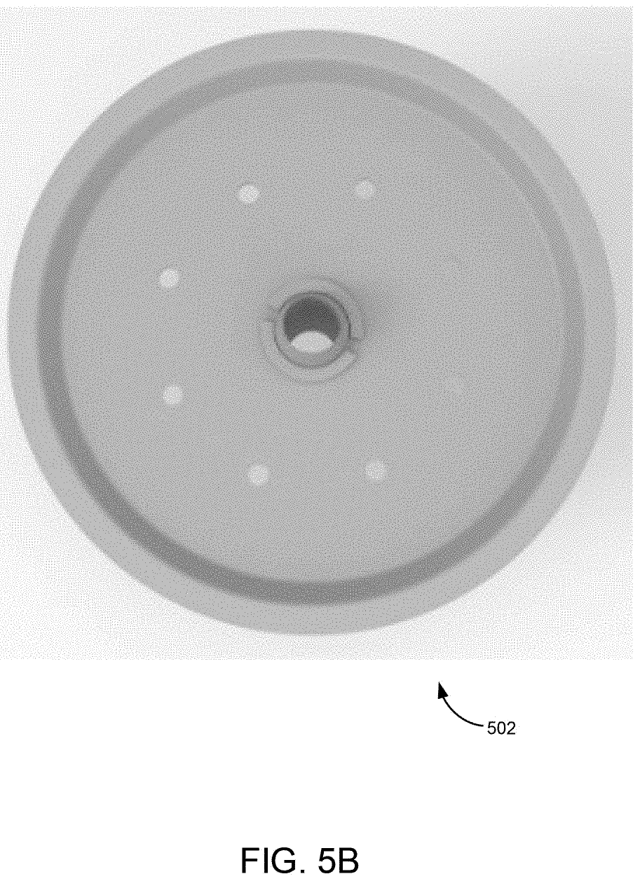

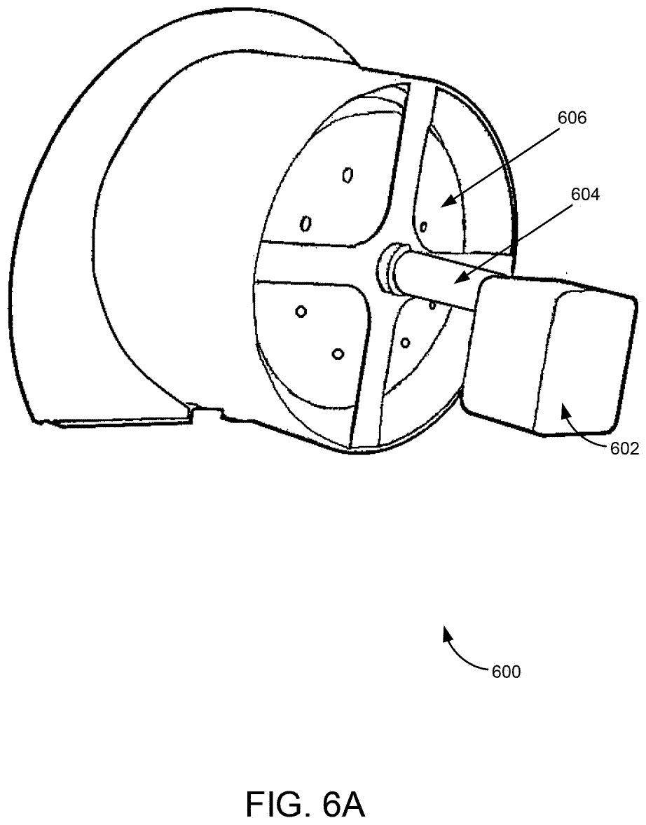

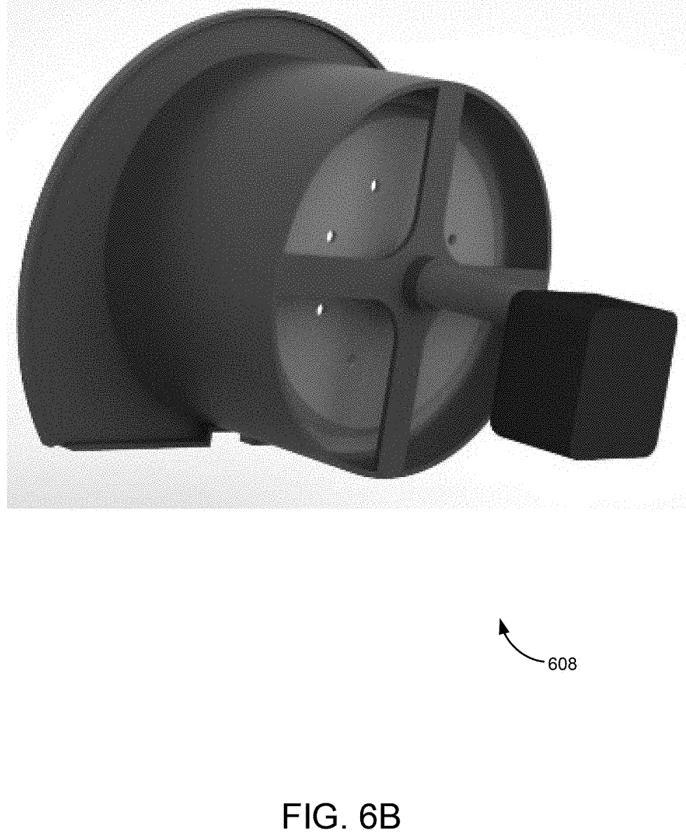

FIG. 5A is an illustrative top view 500 of the watering base according to the embodiment of the present disclosure. The watering base may have a plurality of small holes that may allow liquid to seep through it. FIG. 5B is another depiction 502 of the watering base of FIG. 5A according to the embodiment of the present disclosure. FIG. 6A is an illustration 600 of the bottom view of the second container 104, watering base 606, and the watering unit 602 (cross section depicted as 206 in FIG. 2A) with a tube 604 connecting the watering unit 602 to the watering base 606. The tube 604 is a part of the watering unit 602. Essentially, the watering unit 602 comprises of the tube 604, a water source, and a motor. During operation, the motor receives power through the power port to which the watering unit 602 is connected. The motor may pump water from via the tube 604 to the watering base 606. FIG. 6B is another depiction 608 of the FIG. 6A according to the embodiment of the present disclosure.

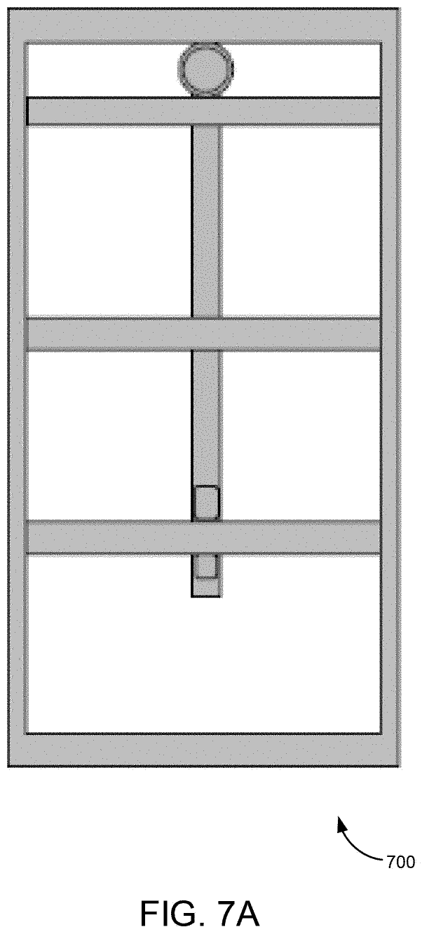

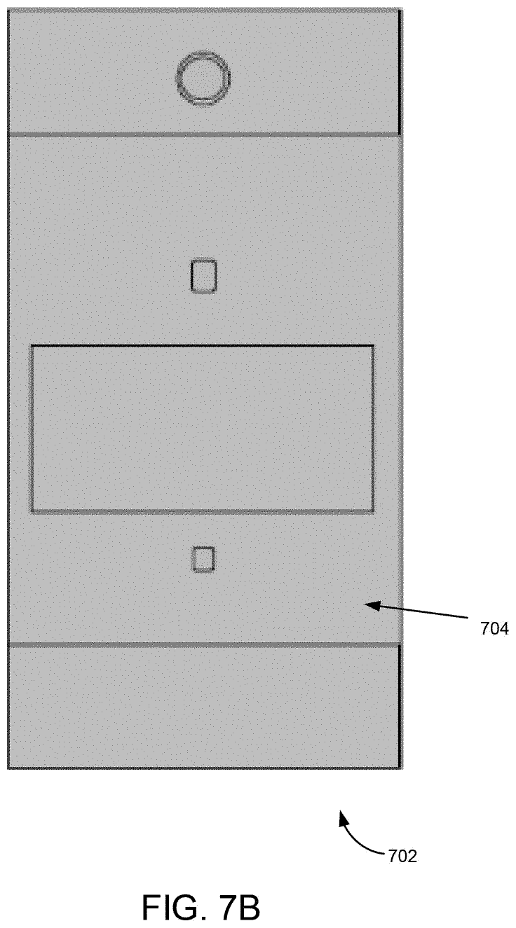

FIG. 7A is an exemplary view of the base structure 700 of the panel of the smart garden station according to the embodiment of the present disclosure. The base structure of the panel may be fitted with an outer body 704 as described in FIG. 7B which is another exemplary view 702 of the panel of the smart garden station. The panel structure may be mounted on Video Electronics Standards Association (VESA) compliant mounts via a plurality of fasteners. Alternatively, they may be fixed onto any custom-made supporting structures with complementing dimensions and openings for fasteners.

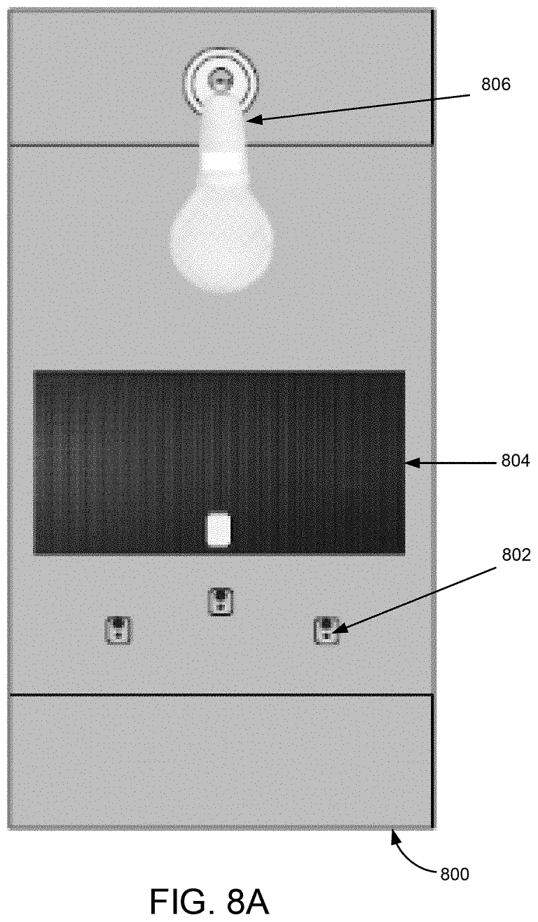

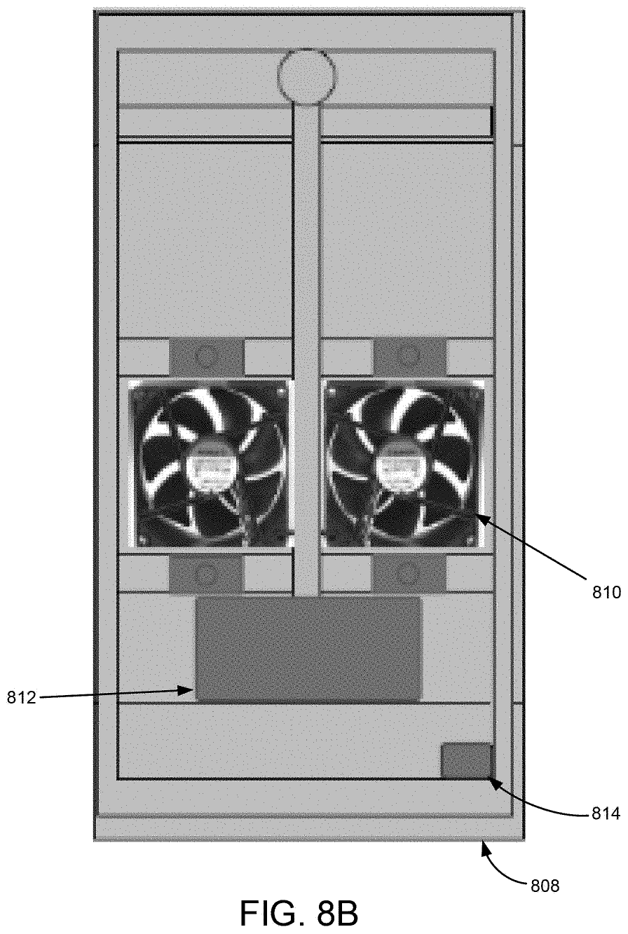

FIG. 8A is an exemplary depiction 800 of the panel of the smart garden station with power ports 802, ventilation unit 804, and lighting unit 806 interfaced to it according to the embodiment of the present disclosure. The panel may be connected to a power source via power cables and the panel may have provisions to have power ports that allows easy interface with lighting units, watering units, etc. The panel may have multiple such power ports depending on its configuration and size. The lighting unit 806 may be connected with a light source as depicted in the figure. Herein, the light source may be a light bulb. Furthermore, the panel may have a processing unit, a memory unit, a set of communication devices, a set of sensors, watering units, etc., mounted or embedded to it. The lighting unit 806 may be configured to provide advantages of full spectrum bulbs. They may further have optical lenses that allows light to get evenly distributed. The luminous efficiency of such a full spectrum light source provides the plants with uniform light which will make the plants grow faster and stay healthier.

FIG. 8B is an exemplary rear view 808 of the smart garden station of FIG. 8A according to the embodiment of the present disclosure. The ventilation unit 804 comprises of a set of fans 810 as described in the figure. The fans may operate at different speeds to control temperature, humidity and/or moisture depending on the requirement of the garden station. Furthermore, a power supply module 812 provides provisions for powering up the different component units of the smart garden station. Input power source (that provides electricity) may be connected to its input power port and a plurality of output ports are provided from it for powering the set of sensors, communication devices, lights, motors, etc. Herein, the output ports refer to the power ports 802 through which different components of the smart garden station are powered. An auxiliary power supply connector 814 is further provided within the panel.

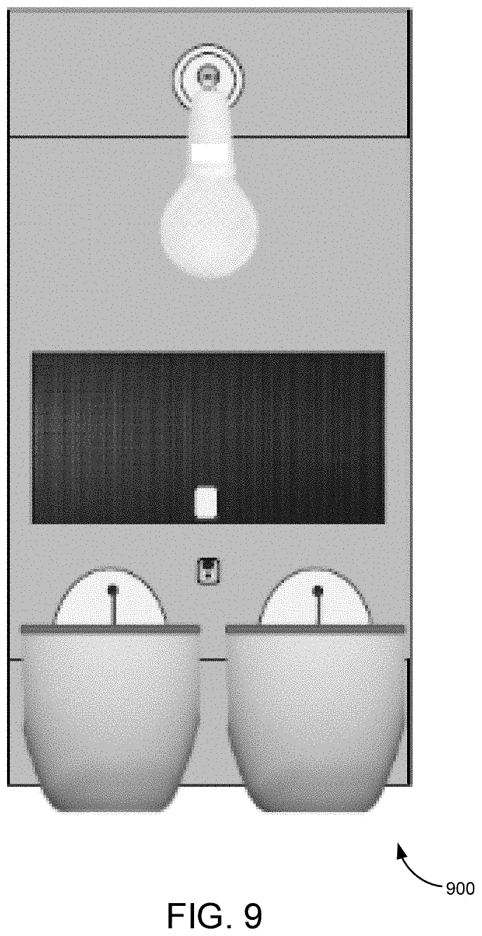

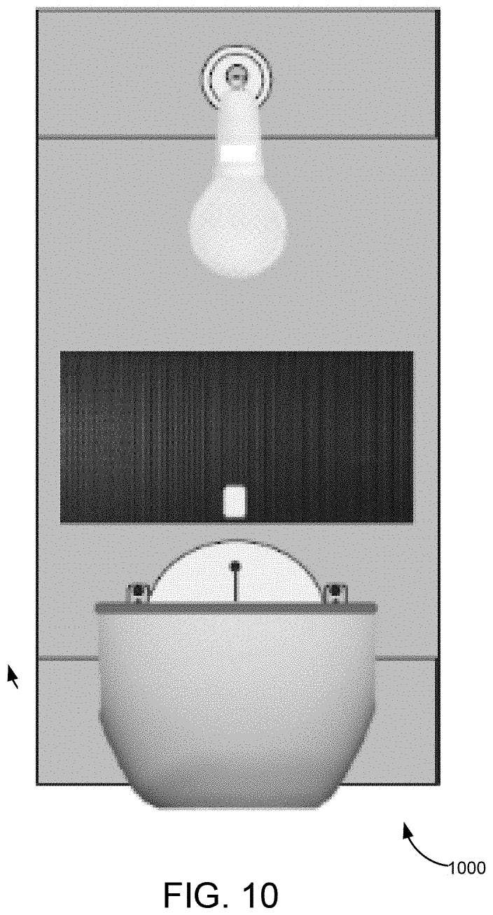

FIG. 9 is an exemplary depiction 900 of a smart garden station with two container pots according to the embodiment of the present disclosure. FIG. 10 is an exemplary depiction 1000 of a smart garden station with a single container pot according to the embodiment of the present disclosure. Depending on the size of the panel, one or more container units (pots) may be connected to each panel,

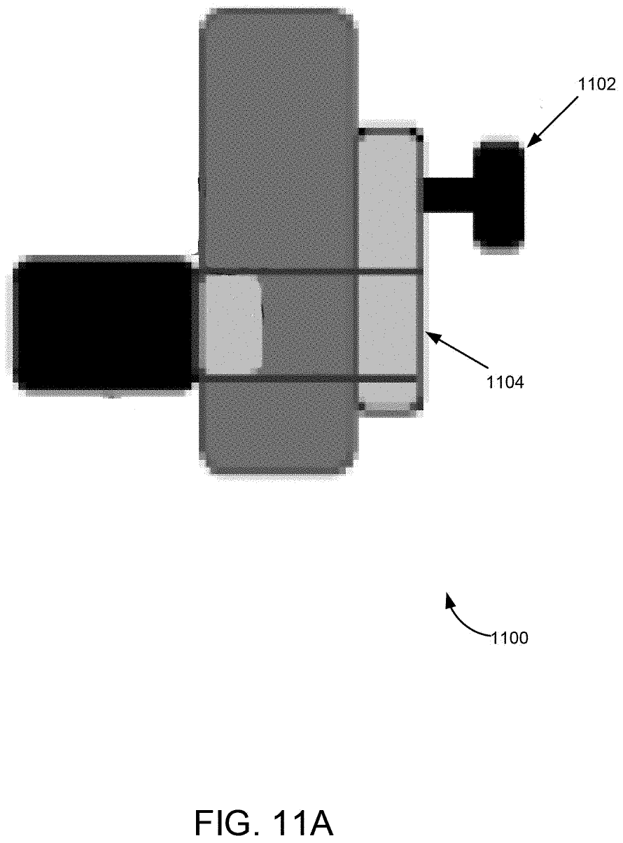

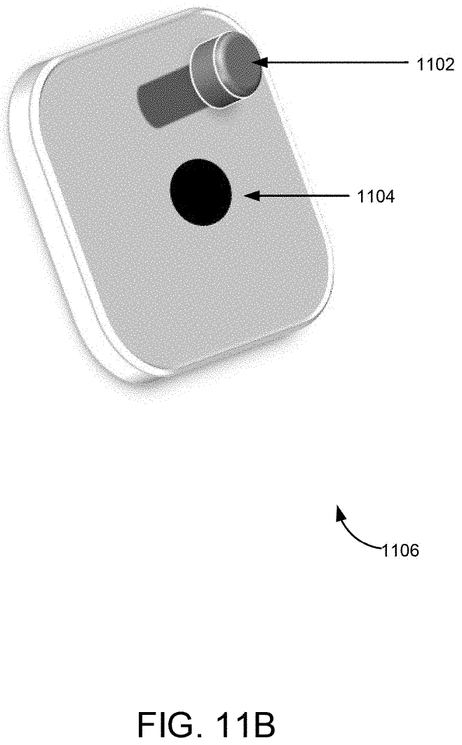

FIG. 11A is a side view 1100 of a power port that may be used in the smart garden station according to the embodiment of the present disclosure. The first container maybe mounted onto the hook 1102 while using the disclosed apparatus. Once mounted onto the panel via hook 1102, the power cables may be connected to the power port insert 1104. In one example, power cables which supply power to the watering unit 602 may be connected to the power port 802 for transferring the power required to operate this component. The watering unit 602 comprises of the motor, the water source, and the tube 604. The motor receives power via the power port 802 through the power cables. This allows the motor to pump water to the watering base 606 via the tube 604.

FIG. 11B is another view 1106 of the power port of FIG. 11A according to the embodiment of the present disclosure. In one example, the power ports and cables may be magnetic in nature. The magnetic power ports may provide more convenience and ease of use during operation of this apparatus. Magnetic power ports may allow easier installation and removal of containers to/from the panel. Furthermore, the hook and connector structure secures the pot container onto the panel more comprehensively.

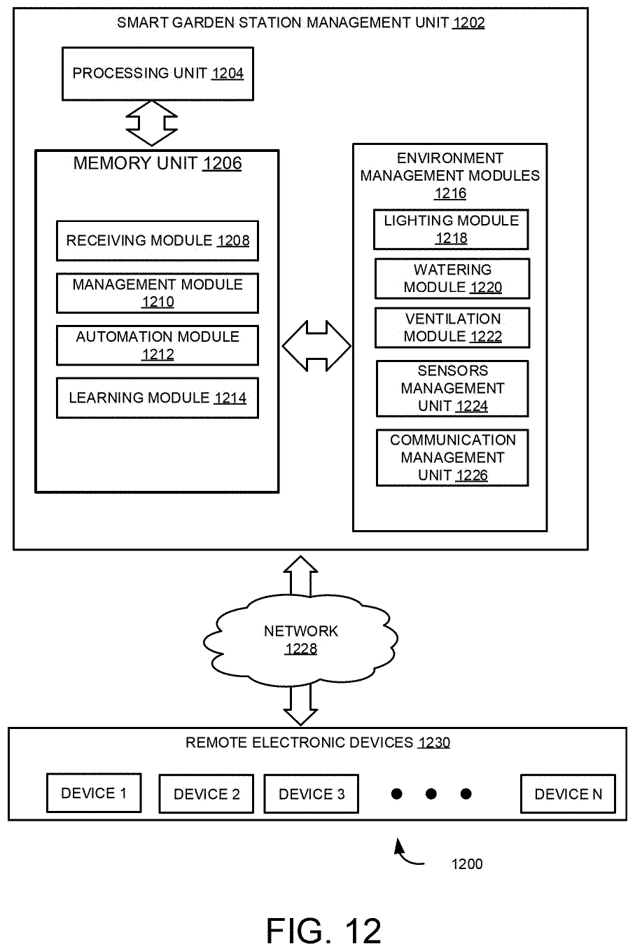

FIG. 12 is a block diagram 1200 of the architecture of the smart garden station management unit according to the embodiment of the present disclosure. Accordingly, the smart garden station management unit 1202 comprises of a processing unit 1204 communicatively couples to a memory unit 1206. The memory unit may comprise of receiving module 1208 for receiving instructions from remote communication devices for monitoring, managing and controlling the smart garden station. The management module 1210 may be responsible for managing the overall function of the garden station such as controlling the working of various components of the apparatus, setting timers, and the like. The automation module 1212 may be configured to automate various processes related to the functioning of the smart garden station. The learning module 1214 may be configured to learn user patterns and store them in memory for future use. The learning module may, in the longer run, work along with the automation module 1212 for providing a self-sustaining garden with least human intervention.

The processor and the memory units may communicate and control the different components of the smart garden station via the environment management modules 1216. These modules comprise a lighting module 1218 that controls the lights around the garden station, a watering module 1220 that controls the motor pump and waters the plant/seeds, a ventilation module 1222 that controls the fans to modulate temperature and humidity, sensors management unit 1224 that continuously reads sensor data, and communication management unit 1226 that manages communication to/from remote electronic devices. These modules and units may be connected to remote electronic devices 1230 via wired/wireless network 1228.

The sensors may be IoT (internet of things) based and may operate wirelessly. They may continuously transmit data to a cloud via internet. Users may be able to access this information from anywhere in the world by accessing this data uploaded to the cloud. It may also provide notifications to the user via calls, text messages, email, and push notifications depending on its configuration.

During operation, seeds or plants belonging to a plurality of species may be planted in a growing medium filled within the second container 104. The growing medium may be soil based or hydroponic based. Instead of soil other growing media may also be used. The lighting unit 806 and the ventilation unit 804 may be mounted on the panel positioned directly above the first and second containers. The set of sensors continuously monitor movement, moisture, humidity, light, temperature, etc., around the smart garden station. The set of communication devices transmit this sensor data to the remote electronic device and for receiving a set of instructions from the remote electronic device.

The remote electronic device generates the set of instructions for providing an optimal environment around the smart garden station for promoting ideal growth of the seeds or the plants based on the data received from the sensors. The electronic device performs this operation by: (i) receiving sensor data from the set of sensors. (ii) processing the received sensor data to determine the environmental conditions around the smart garden station such as growth pattern of the plants/seeds, amount of light emitted by the lighting unit, temperature, humidity in the air, and water content in the growing medium, (iii) comparing the determined environmental conditions with a pre-determined benchmark to determine ideal environmental conditions, (iv) generating the set of instructions to provide the ideal environmental conditions around the smart garden station, wherein the set of instructions are executed by the processing unit.

The processing unit communicatively coupled with the memory unit is configured to manage the set of communication devices, the set of sensors, the lighting unit, the watering unit, and the ventilation unit. Upon receiving the abovementioned set of instructions, the processing unit controls the environment around the smart garden station by controlling the lighting unit, watering unit, and the ventilation unit.

The smart garden station further comprises power cables to provide power for the functioning of the smart garden station, wired or wireless network connectivity for connecting the set of communication devices with the remote electronic device, power ports, and network ports. It is pertinent to note that the central panel of the smart garden station acts as an interface for the components of the smart garden station including the first container, the ventilation unit, the lighting unit, the set of sensors, the set of communication devices, the memory unit, the processing unit, power ports, network ports, and power cables. The panel may be mounted onto VESA (Video Electronics Standards Association) compliant wall mounts or custom-made supporting mounts.

Furthermore, the remote electronic device that receive data and provide set of instructions to control the smart garden station may include a personal computer, a laptop computer, a table computer, a smartphone, or a smartwatch. The remote electronic device may be able to monitor and control the environmental conditions of the smart garden station via a web-based application. An application may be installed onto the device for the same or a web-browser may be used to access and control the station.

The electronic device may generate the set of instructions for controlling the environment around the smart garden station automatically or manually with the help of a user. A user may be provided complete control over managing various components of the garden station via the electronic device. Alternatively, the electronic device may be configured to automatically generate the set of instructions to manage the garden station thereby making it a “smart” garden station.

In another embodiment, the smart garden station may further comprise a camera unit that continuously transmits video feed of the smart garden station to the remote electronic device via the set of communication devices. A user may even visually monitor and manage the growth of the plants/seeds in this case.

Components of smart garden station management unit 1202 may be any combination of hardware and programming to implement the functionalities described herein. In some implementations, the programming may be processor executable instructions stored on a non-transitory machine-readable storage medium (e.g., memory), and the hardware may include at least one processing resource to retrieve and/or execute those instructions. Processing unit 1204 may comprise processor(s) that may include, but are not limited to, one or more digital signal processors (DSPs), one or more microprocessor, one or more special-purpose computer chips, one or more field-programmable gate arrays (FPGAs), one or more application-specific integrated circuits (ASICs), one or more computer(s), various analog to digital converters, digital to analog converters, and/or other support circuits. Processor(s) thus may also include the functionality to encode messages and/or data or information. Processor(s) may include, among other things, a clock, an arithmetic logic unit (ALU) and logic gates configured to support operation of processor(s). Further, the processor(s) may include functionality to execute one or more software programs, which may be stored in the memory or otherwise accessible to processor(s).

Memory Unit 1206, may store any number of pieces of information, and data, used by the system to implement the functions of the system. The memory may include for example, volatile memory and/or non-volatile memory. Examples of volatile memory may include but are not limited to volatile random-access memory (RAM). The non-volatile memory may additionally or alternatively comprise an electrically erasable programmable read only memory (EEPROM), flash memory, hard drive, and the like. Some examples of the volatile memory include, but are not limited to, dynamic RAM, static RAM, and the like. Some example of the non-volatile memory includes, but are not limited to, hard disks, magnetic tapes, optical disks, programmable read only memory, erasable programmable read only memory, electrically erasable programmable read only memory, flash memory, and the like. Memory 106 may be configured to store information, data, applications, instructions or the like for enabling the system to carry out various functions in accordance with various example embodiments. Additionally, or alternatively, the memory unit 1208 may be configured to store instructions which when executed by processor(s) causes the system to behave in a manner as described in various embodiments.

In one implementation, the network 1228 may be a wireless network, a wired network or a combination thereof. The network 1228 may be implemented as one of the several types of networks, such as intranet, local area network (LAN), wide area network (WAN), the internet, and the like. The network 1228 may either be a dedicated network or a shared network. The shared network represents an association of the several types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the network 1228 may include a variety of network devices, including routers, bridges, servers, computing devices, storage devices, and the like.

Some or all of the system components and/or data structures may also be stored as contents (e.g., as executable or other machine-readable software instructions or structured data) on a non-transitory computer-readable medium (e.g., as a hard disk, a computer memory; a computer network or cellular wireless network or other data transmission medium; or a portable media article to be read by an appropriate drive or via an appropriate connection, such as a DVD or flash memory device) so as to enable or configure the computer-readable medium and/or one or more host computing systems or devices to execute or otherwise use or provide the contents to perform at least some of the described techniques. Some or all of the components and/or data structures may be stored on tangible, non-transitory storage mediums. Some or all of the system components and data structures may also be provided as data signals (e.g., by being encoded as part of a carrier wave or included as part of an analog or digital propagated signal) on a variety of computer-readable transmission mediums, which are then transmitted, including across wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of this disclosure may be practiced with other computer system configurations.

It may be noted that the above-described examples of the present solution are for the purpose of illustration only. Although the solution has been described in conjunction with a specific embodiment thereof, numerous modifications may be possible without materially departing from the teachings and advantages of the subject matter described herein. Other substitutions, modifications and changes may be made without departing from the spirit of the present solution. All the features disclosed in this specification (including any accompanying claims, abstract and drawings), and/or all of the steps of any method or process so disclosed, may be combined in any combination, except combinations where at least some of such features and/or steps are mutually exclusive.

The terms “include,” “have,” and variations thereof, as used herein, have the same meaning as the term “comprise” or appropriate variation thereof. Furthermore, the term “based on”, as used herein, means “based at least in part on.” Thus, a feature that is described as based on some stimulus can be based on the stimulus or a combination of stimuli including the stimulus.

The present description has been shown and described with reference to the foregoing examples. It is understood, however, that other forms, details, and examples can be made without departing from the spirit and scope of the present subject matter that is defined in the following claims.

What is claimed is:

1. A smart garden station comprising:
a first container with an open top side that can be mounted on a panel;
a second container that can be inserted into the first container, wherein the second container when placed inside the first container, fills the top portion of the first container and leaves a space at the bottom portion of the first container, and wherein the second container is fully open at its top side and partially open at its bottom side;
a watering base with a plurality of openings placed above the partially open bottom side of the second container, wherein the watering base is connected to a watering unit disposed within the bottom portion of the first container via a tube;
seeds or plants planted in a growing medium disposed in the second container;
a lighting unit and a ventilation unit mounted on the panel and positioned directly above the first and second containers;
a set of sensors for continuously monitoring movement, moisture, light, humidity, and temperature around the first and second containers;
a set of communication devices for transmitting sensor data generated by the set of sensors to a remote electronic device and for receiving a set of instructions from the remote electronic device, wherein the electronic device generates the set of instructions for providing an optimal environment around the smart garden station for promoting ideal growth of the seeds or the plants based on the data received from the sensors; and
a processing unit communicatively coupled with a memory unit, wherein the processing unit is configured to manage the set of communication devices, the set of sensors, the lighting unit, the watering unit, and the ventilation unit, wherein the processing unit controls the environment around the smart garden station by controlling the lighting unit, watering unit, and the ventilation unit based on the set of instructions received from the remote electronic device.

2. The smart garden station of claim 1 further comprises power cables to provide power for the functioning of the smart garden station, wired or wireless network connectivity for connecting the set of communication devices with the remote electronic device, power ports, and network ports.

3. The smart garden station of claim 2, wherein the panel acts as a central structure that acts as an interface for the components of the smart garden station including the first container, the ventilation unit, the lighting unit, the set of sensors, the set of communication devices, the memory unit, the processing unit, power ports, network ports, and power cables.

4. The smart garden station of claim 1, wherein the panel can be mounted onto VESA (Video Electronics Standards Association) compliant wall mounts or custom-made supporting mounts.

5. The smart garden station of claim 1, wherein the remote electronic device may include at least one of: a personal computer, a laptop computer, a tablet computer, a smartphone, or a smartwatch.

6. The smart garden station of claim 5, wherein the remote electronic device can monitor and control the environmental conditions of the smart garden station via a web-based application.

7. The smart garden station of claim 1, wherein the electronic device generates the set of instructions for controlling the environment around the smart garden station automatically or with the help of a user.

8. The smart garden station of claim 1, wherein the electronic device can be configured to automatically generate the set of instructions by:
receiving sensor data from the set of sensors;
processing the received sensor data to determine the environmental conditions around the smart garden station such as growth pattern of the plants/seeds, amount of light emitted by the lighting unit, temperature, humidity in the air, and water content in the growing medium;
comparing the determined environmental conditions with a pre-determined benchmark to determine ideal environmental conditions; and
generating the set of instructions to provide the ideal environmental conditions around the smart garden station, wherein the set of instructions are executed by the processing unit.

9. The smart garden station of claim 1, wherein the ventilation unit comprises of a set of fans.

10. The smart garden station of claim 1, wherein the watering unit comprises of a motor, a water source, and a tube that connects the water source with the watering base fixed on the base of the second container.

11. The smart garden station of claim 10, wherein the watering unit receives power though the power cables connected to the power port and allows the motor to pump water to the watering base via the tube.

12. The smart garden station of claim 1 further comprises a camera unit that continuously transmits video feed of the smart garden station to the remote electronic device via the set of communication devices.

13. The smart garden station of claim 1, wherein the panel comprises of one or more hooks using which the first container is mounted on the panel.

14. The smart garden station of claim 12, wherein power ports are provided below the one or more hooks onto which the power cable that powers the components of the first container and the second container are inserted.

15. The smart garden station of claim 13, wherein the combination of hooks and power cables connected to the power ports securely supports the first container mounted on the panel.
